(19) United States
(12) Patent Application Publication
(10) Pub. No.: US 2008/0319041 A1
(43) Pub. Date: Dec. 25, 2008

(54) Lofexidine enantiomers for use as a treatment for CNS disease and pathologies and its chiral synthesis

(76) Inventors: George Digenis, Louisville, KY (US); Peter A. Crooks, Nicholasville, KY (US); Abeer Al-Ghananeem, Lexington, KY (US)

Correspondence Address: FROST BROWN TODD, LLC, 2200 PNC CENTER, 201 E. FIFTH STREET, CINCINNATI, OH, 45202, US

(21) Appl. No.: 11/376,710

(22) Filed: Mar. 14, 2006

Related U.S. Application Data
(60) Provisional application No. 60/661,525, filed on Mar. 14, 2005.

(51) Int. Cl. A61K 31/4164; C07D 233/22

(52) U.S. Cl. 514/401; 548/353.1

(57) ABSTRACT

The invention relates to methods for treatment of CNS disease and pathologies using non-racemic mixtures of lofexidine enantiomers. The invention also relates to processes for the manufacture of chirally pure enantiomers of lofexidine.

### CROSS REFERENCE

This application claims the benefit of Provisional Patent Aplication Ser. No. 60661,525 filed Mar. 14, 2005.

### FIELD OF THE INVENTION

The invention relates to methods for treatment of CNS disease and pathologies. More particularly, the invention relates to methods for the treatment of CNS disease and pathologies by administering Lofexidine enantiomers. Most particularly, the invention relates to methods of treatment of CNS disease, such as opioid detoxification, with less undesirable side effects than conventional treatments. This invention also relates to a novel chiral synthesis of Lofexidine enantiomers.

### BACKGROUND OF THE INVENTION

Opioid addiction is a serious public health concern in the United States (US). Heroin has been reported to be the most prominent illicit drug of abuse among admissions at publicly-funded substance abuse treatment facilities in the US. At some time in their lives, about 2.4 million people have used heroin; in 1997, there were 81,000 new heroin users of whom 87% were less than 26 years of age. In spite of efforts to decrease illicit drug abuse, the problem escalates and the abusing population is increasingly younger. Hospital emergency room episodes from 21 metropolitan areas show that 14% of drug-related emergency room episodes involved heroin, and such episodes increased more than 2-fold from 1991 to 1996. Additionally, prescription opioid abuse escalates; the number of people addicted to prescription pain relievers is 3-fold higher than those addicted to heroin. For example, from 1999 to 2001, the non-medical use of OxyContin® increased 4-fold, and its use continues to escalate.

Generally, opioid addiction has been associated with high morbidity and mortality, with a 15-20 fold increase in risk of death for intravenous drug users compared with their same age peers. Clearly, the medical and social importance of the development of effective treatments for opioid addiction is well recognized. Surprisingly, few treatment options for opioid addiction are available.

Withdrawal, maintenance and relapse are considered the progressive stages for treatment of opioid addiction. There are two predominant management strategies for the treatment of opioid addiction, detoxification and substitution therapy, which are typically combined with medical, social and psychological support. A majority of individuals may benefit from remaining in the maintenance phase for an indefinite period of time, while others may be able to directly undergo medically-supervised detoxification and/or relapse therapy, without the need for maintenance therapy. Methadone and buprenorphine constitute the most commonly used pharmacotherapies. Although patients continue to be successfully treated with methadone, a mμopioid receptor agonist, several disadvantages of methadone treatment include the length of time for withdrawal, the difficulty of obtaining complete abstinence, and liability for its abuse. Due to the abuse liability of methadone and its consequent Schedule II classification by the Drug Enforcement Administration (DEA), methadone has additional disadvantages with respect to its prescription requirements, the carefully controlled conditions under which it is dispensed, and the annoyance experienced by patients who must frequently visit the dispensing unit to obtain their methadone dosages.

BritLofex™ (Lofexidine hydrochloride 0.2 mg tablet), an α₂-adrenergic agonist, is used as a non-opioid medication for opioid detoxification in the United Kingdom (UK). There is no non-opioid medication approved by the Food and Drug Administration (FDA) for this indication in the US. The only medications currently approved by the FDA for opioid detoxification are methadone and buprenorphine, both opioid receptor agonists and both associated with abuse liability. Clonidine, an α₂-adrenergic agonist, is often used “off-label” for this indication in the U.S. However, clonidine has not been approved by the FDA for this indication. However, the use of clonidine is limited by its side-effect profile, i.e., significant hypotension at doses effective in alleviating opioid withdrawal symptoms.

In contrast, Lofexidine HCl is the only non-opiate, non-addictive treatment approved for use in the UK to manage withdrawal symptoms in patients undergoing opiate detoxification. Lofexidine has been found to be effective in reducing the symptoms associated with heroin withdrawal such as chills, vomiting, sweating, stomach cramps, diarrhea, muscle pain, and runny nose and eyes. In the UK, the treatment is responsible for approximately 20,000 detoxifications per year. The drug's proven level of safety permits its use in an outpatient situation. This is of great importance to patients in the US who are located in parts of the country where treatment clinics are not readily available.

Although naltrexone, methadone and more recently buprenorphine are FDA approved in the treatment of opioid addiction, these opioid treatments are associated with high relapse rates. Furthermore, there is currently insufficient availability of methadone and buprenorphine treatment for patients who abuse opioids. A significant number of these patients are undergoing detoxification treatments. However, the great risk of abuse and several other existing restrictions, such as medical prescribing and pharmaceutical dispensing, limit the use of methadone and buprenorphine for outpatient detoxification. In addition, the unapproved status of clonidine, its side effects, such as the lowering of blood pressure, and moderate efficacy limit its use. A substantial amount of research is ongoing to understand the mechanisms that may underline the high rates of relapse associated with opioid addiction. There is growing evidence that chronic drug use results in neuroadaptive changes in brain stress and reward circuits that may be associated with increased drug craving and risk of relapse particularly in the face of environmental triggers such as stressful life events and drug cues.

The lofexidine hydrochloride tablets available in the UK market (BritLofex™) contain the racemic mixture of the drug. However, since lofexidine enantiomers exhibit different affinities for central the nervous system neurotransmitter receptors involved in (±)-lofexidine's action as a medication for opioid detoxification, each of these enantiomers may have therapeutic benefits in the treatment of opioid addiction.

### SUMMARY OF THE INVENTION

To maximize the effect of enantiomers of Lofexidine, (−)-lofexidine and (+)-lofexidine can be used in the treatment of opioid addiction and other related drug addictions. In addition, the use of non-racemic mixtures of (−)-lofexidine and (+)-lofexidine (i.e. mixtures where the molar ratio of one enantiomer is greater than, or less than, that of the other) can further benefit the patient in different ways.

(−)-Lofexidine is more potent than (+)-lofexidine at brain adrenergic receptors involved in the mechanism of opioid detoxification. Thus, lower doses of (−)-lofexidine can be used, compared to those used for (±) Lofexidine. On the other hand, (+)-lofexidine may also have advantages over (±)Lofexidine as an opioid detoxification agent since it has practically no effect on reducing blood pressure or bradycardiac activity. Additionally, the (+)-lofexidine enantiomer can be combined with a small proportion of the centrally active (−)-lofexidine to afford a product formulation with a lower effective dose with reduced undesirable side effects.

This invention also relates to a physical separation and a synthetic procedure for producing large quantities of either enantiomer by a preparative manufacturing-scale procedure.

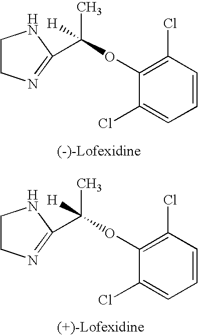

### DETAILED DESCRIPTION OF THE PREFERRED EMBODIMENT

Preliminary studies have shown that the enantiomers of lofexidine exhibit different interacting affinity with α₂-adrenergic receptor. α-Adrenoreceptor activity of lofexidine is believed to reside predominantly in the (−)-enantiomer. It possesses about nine times higher affinity than the (+)-enantiomer for the α₂-adrenergic binding sites in rat brain membranes. The (−)-enantiomer also exhibits about four times greater affinity than the (+)-enantiomer for α₁-adrenergic receptors. Other studies demonstrate in pithed normotensive rats, intravenous administered (−)-lofexidine elicits pressor effects at doses 20 times lower than similarly administered (+)-lofexidine. Besides, following intravenous administration to pentobarbitone anesthetized normotensive rats (−)-lofexidine is twenty times more effective than (+)-lofexidine in decreasing mean arterial pressure and heart rate. (−)-Lofexidine was also found to be thirty times more potent than the (+)-lofexidine in decreasing the increased heart rate evoked by electrical stimulation in the pithed rat. Similarly, the electrically stimulation-induced increasing in diastolic pressure also was found to be more effectively unpaired by (−)-lofexidine.

Since lofexidine enantiomers exhibit different affinities for central nervous system neurotransmitter receptors, (±)-lofexidine's action as a medication for opioid detoxification, may have therapeutic benefits in the treatment of opioid addiction. The use of both (−)-lofexidine and (+)-lofexidine in the treatment of opioid addiction and other related drug addictions may offer additional benefits over the use of racemic (±)-lofexidine. In addition, the use of mixtures of (−)-lofexidine and (+)-lofexidine that are in a molar ratio of greater than, or less than one, but not equimolar (i.e. a racemic mixture) can be use in the treatment of opioid addition to minimize undesirable side effects. Additionally, this invention is directed at a physical separation and a synthetic procedure for producing large quantities of either lofexidine enantiomer by a preparative manufacturing-scale procedure.

(−)-Lofexidine is a more suitable therapeutic agent than (±)-lofexidine because it is more potent than (+)-lofexidine at brain adrenergic receptors involved in the mechanism of opioid detoxification. Thus, lower doses of (−)-lofexidine can be used, compared to those used for (±) Lofexidine, reducing unwanted peripheral pressor effects of (±)-lofexidine given at higher doses.

(+)-lofexidine also has advantages over (±)-Lofexidine as an opioid detoxification agent since it has practically no effect on reducing blood pressure, and is only four times less potent than (−)-lofexidine at α₁-adrenergic receptors in rat brain membranes, and nine times less potent than (−)-lofexidine at α₂-adrenergic receptors in rat brain membranes. Thus, although the dose of (+)-lofexidine compared to (±)-lofexidine may need to be increased due to this reduced affinity for the CNS receptors, the (+)-enantiomer exhibits considerably lower peripheral side effects on blood pressure. Additionally, the (+)-lofexidine enantiomer can be combined with a small proportion of the centrally active (−)-lofexidine to afford a product formulation with a lower effective dose with reduced undesirable side effects. The amount of the more active (−)-lofexidine in the optimal non-racemic mixture should be of such amount that it will not activate peripheral adrenergic receptors causing undesirable side effects such as, blood pressure lowering, bradycardiac activity etc. Thus, in summary, the use of the individual (+)-lofexidine and (−)-lofexidine enantiomers, and also non-racemic mixtures of these two enantiomers will produce less undesirable peripheral side effects than the use of a racemic mixture.

This invention relates to novel uses of (−)-lofexidine, (+)-lofexidine, and a non-racemic mixture of lofexidine enantiomers as a treatment to relieve symptoms in patients undergoing opiate detoxification, to decrease stress-induced reinstatement of seeking addictive materials, to treat cardiovascular complications in patients with obstructive sleep apnea, to treat chronic pelvic pain in females as well as pain management in general such as migraine and neuropathic pain, to treat behavioral disorders (i.e. attention-deficit/hyperactivity disorder (ADHD)), to prevent adverse effects of N-methyl-D-aspartate (NMDA) antagonists or schizophrenia-associated (NMDA) receptor hypofunction, to treat intraocular pressure (IOP), to alleviate tobacco and alcohol withdrawal symptoms, and as an antidiarrheal agent. This invention further relates to novel use of (−)-lofexidine and (+)-lofexidine as growth-enhancing agent in livestock feeds.

The present invention also relates to processes for the stereo specific synthesis and a physical separation process of resolution of (−)-lofexidine and (+)-lofexidine. The processes for the preparation of enantiomerically pure R-(+) or S-(−)-lofexidine (2-[1-(2,6-dichlorophenoxy)ethyl]-4,5-dihydro-1H-Imidazole) or pharmaceutically acceptable salts thereof by resolution of (R,S)-lofexidine hydrochloride with Di-p-toluoyl-D-tartaric acid and S-(+)-mandelic to form a mixture of diastereomeric salts, separating these salts by kinetic resolution in a mixture of solvent systems of the kind such as herein described, in the specified time and temperature range to provide said R-(+)-lofexidine hydrochloride or S-(−)-lofexidine hydrochloride with excellent chiral purity more than 99.9%. More particularly, it relates to the preparation of pure lofexidine hydrochloride.

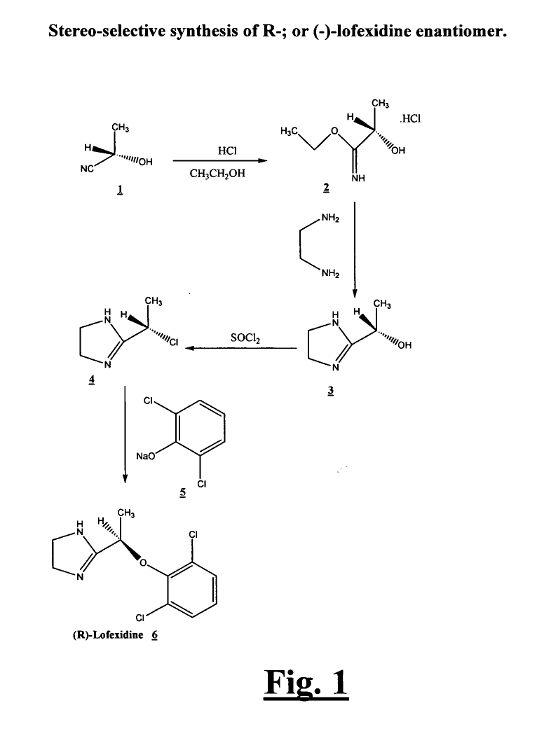

This novel process for preparing (−)-lofexidine and (+)-lofexidine comprises:

[a] Reacting a racemic form of lofexidine with an aliphatic or aromatic (+)-chiral acid or an aliphatic or aromatic (−) chiral acid (such as but not limited to: tartarate, lactate, citrate, mandelate, fumerate, citrate, abscisic acid, 3-hydroxyisobutyric acid, cholic acid, deoxycholic acid, aminoacids, glycocholic acid and related steroid carboxylic acids) in order to form a mixture of the (+)(−) and (+)(+) diastereomeric lofexidine salts, or (−)(−) and (−)(+) diastereomeric lofexidine salts, respectively;

[b] Separating the diastereomeric salts i.e.: (+)(−) lofexidine salt from the (+)(+) lofexidine salt, or the (−)(−) lofexidine salt from the (−)(+) lofexidine salt by a process of fractional crystallization; or by a preparative chromatographic process or preferential adsorption method;

[c] Treating the (+)(−) lofexidine salt or the (−)(−) lofexidine salt so obtained with base to liberate (−)-lofexidine;

[d] Treating the (+)(+) lofexidine salt or the (−)(+) lofexidine salt so obtained with base to liberate (+)-lofexidine; and

[e] Utilizing a chiral chromatographic matrix to separate a racemic mixture of lofexidine into its component enantiomers by a process of preparative chromatography to obtain optically pure (−)-lofexidine and optically pure (+)-lofexidine;

[f] Separating a racemic mixture of lofexidine into its component enantiomers by a process of chemical derivatization with a chiral acylating agent, separating the two resulting diastereomeric N-acyl lofexidine isomers by either fractional crystallization or non-chiral preparative chromatography, and treating the isolated diastereomeric N-acyl lofexidine analogs with base to generate optically pure (−)-lofexidine and optically pure (+)-lofexidine.

[g] Carrying out a chiral synthetic process (see FIG. 1) for the production of the (+) and (−)-enantiomers of lofexidine, comprising the following steps:

1, S-lactonitrile 1 is added to ethanol under acidic condition (hydrochloric acid) in order to form ethyl lactimidate hydrochloride 2, ethylene diamine is then added in sufficient amount in order to form 2-(1-hydroxy-ethyl)-2-imidazoline 3. the conversion of 2-(1-hydroxy-ethyl)-2-imidazoline 3 to the alkyl halide 4 is conducted by treatment of 3 with thionyl chloride through a Sni mechanism which retains chirality. The resulting S-2-(1-chloride-ethyl)-2-imidazoline 4 is reacted with 2,6-dichlorophenol sodium salt 5 to form R-2-[1-2,6-dichlorophenoxy)-ethyl]-1,3-diazacyclopent-2-ene) 6 also known as R-2-[1-(2,6-dichlorophenoxy)-ethyl] imidazoline which corresponds to R-lofexidine. This reaction occurs with complete chiral inversion. Subsequently, a hydrochloride salt of the enantiomer is formed. FIG. 1 shows the synthesis of R-; or (−)-lofexidine enantiomer starting with pure chiral form of S-; or (+)-lactonitrile, the same process is carried out for the formation of S-; or (+)-lofexidine enantiomer using R-; or (−)-lactonitrile.

[h] Converting the enantiomerically pure free base of lofexidine so obtained with an appropriate acid so as to obtain a pharmaceutically acceptable salt thereof.

### Experimental

1) Resolution of (−)-lofexidine and (+)-lofexidine enantiomers found in the racemic mixture using chiral stationary phases by HPLC method:

A chiral chromatographic matrix was used to separate a racemic mixture of lofexidine into its component enantiomers by a process of HPLC to obtain optically pure (−)-lofexidine and optically pure (+)-lofexidine. The separation was performed using a chiral stationary phase consisted of D-glucose cyclodextran complex (Cyclobond HP-RSP) from Astec Company (Whippany, N.J., USA) using a mobile phase consisted of 10 mM ammonium acetate (88%), acetonitrile (8%), and methanol (8%) at 0.85 ml/min flow rate. Analysis was performed using Agilent series 1100 HPLC system comprising a solvent degasser unit, quaternary pump, autosampler, and DAD detector. Using such chiral stationary phase in a preparative scale enables the yield of gram quantities of desired enantiomers.

Resolution of (−)-lofexidine and (+)-lofexidine enantiomers found in the racemic mixture using a chiral acid, not only diastereomeric salt formation but also preferential crystallization:

Optical resolution of (±)-lofexidine hydrochloride by using the classical methods of salt formation with a chiral acid such as, [(Di-p-toluoyl-D-tartaric acid [□]D²⁰+142° (c=1, CH₃OH)] as shown in FIG. 1, yielded (−)-lofexidine hydrochloride and (+)-lofexidine hydrochloride enantiomers (yield=87%). The method comprised the following steps:

A racemic form of lofexidine (10 mmol) was placed in ethanol (100 mL), and the chiral acid (+)-Di-p-toluoyl-D-tartaric acid was added in order to form a mixture of the (+)(−) and (+)(+) diastereomeric lofexidine salts. The diastereomeric salts i.e.: (+)(−) lofexidine Di-p-toluoyl-D-tartarate salt was separated from the (+)(+) lofexidine Di-p-toluoyl-D-tartarate salt by a process of fractional crystallization. 10 mL methanol and 1 ml water was added and the mixture was heated for 1 hour at 55-65° C. After the mixture became clear it was left to cool down at room temperature. The crystals were isolated after two days, dried under vacuum. Recrystallization was performed using ethanol (20 volumes). Final yield was 87%.

Chiral purity of the resulting crystals was tested by the chiral HPLC method. The (+)(−) lofexidine Di-p-toluoyl-D-tartarate salt or the(+)(+) lofexidine Di-p-toluoyl-D-tartarate salt obtained was treated with a base such as 0.1 N sodium carbonate to liberate (−)-lofexidine and (+)-lofexidine. The resulting enantiomerically pure free base of (−)-lofexidine and (+)-lofexidine was converted to lofexidine hydrochloride salt.

We claim:

1. A method of treating central nervous system disease and pathologies comprising:
administering lofexidine where the molar ratio of (−)-lofexidine to (+)-lofexidine is not one.

2. A process for the manufacture of highly optical pure R-(−) or S-(+)-lofexidine hydrochloride which comprising the steps of: (a) resolving (R, S)-lofexidine with an organic acid to form a mixture of diastereomeric salts (b) subjecting the diastereomeric salts to a solvent system (c) separating the diastereomeric salt after crystallization by filtration; (d) liberating optically pure R-(−) or S-(+)-lofexidine free base and formation of a hydrochloride salt with optical purity more than 99.9%.

3. A process as claimed in claim 2 wherein said organic solvent is Di-p-toluoyl-D-tartaric acid.

4. A process as claimed in claim 3 wherein Di-p-toluoyl-D-tartaric acid is employed in 1-1.5 molar ratio with that of (R, S)-lofexidine.
